(19) United States
(12) Patent Application Publication
(10) Pub. No.: US 2002/0164342 A1
(43) Pub. Date: Nov. 7, 2002

(54) Recombinant cat allergen, Fel dI, expressed in baculovirus for diagnosis and treatment of cat allergy

(76) Inventors: Paul M. Guyre, Hanover, NH (US); Joel Goldstein, Piscataway, NJ (US); Zining Wu, Collegeville, PA (US); Amanda Wanwen Sun, Collegeville, PA (US)

Correspondence Address: Licata & Tyrrell P.C., 66 E. Main Street, Marlton, NJ, 08053, US

(21) Appl. No.: 10/054,444

(22) Filed: Jan. 22, 2002

Related U.S. Application Data
(63) Continuation of application No. 09/410,963, filed on Oct. 5, 1999.
(60) Provisional application No. 60/103,284, filed on Oct. 6, 1998.

(51) Int. Cl. A61K 39/00; G01N 33/53; G01N 33/567; C12N 7/00; C12N 15/866

(52) U.S. Cl. 424/184.1; 435/7.2; 435/456; 435/235.1

(57) ABSTRACT

Recombinant Fel dI cat allergens expressed in baculovirus for diagnosis and treatment of allergy to cats in humans are provided.

This application is a continuation of U.S. patent application Ser. No. 09/410,963 filed Oct. 5, 1999 and claims the benefit of U.S. Provisional Application No. 60/103,284, filed Oct. 6, 1998.

This invention was made in the course of research sponsored by the National Institutes of Health. The U.S. Government may have certain rights in this invention.

### BACKGROUND OF THE INVENTION

Fel dI is the major allergen from cats. Natural Fel dI consists of two polypeptide chains, chain 1(ch1) and chain 2(ch2) which are normally linked by a disulfide bond. Fel dI has been cloned and sequenced. However, the immunoreactivity of rFel dI chains expressed in bacteria is not comparable to that of the natural allergen (Shint et al. JACI 1995,1221).

### SUMMARY OF THE INVENTION

An object of the present invention is to provide a composition for diagnosis and treatment of cat allergy in humans comprising a baculovirus expressed recombinant Fel dI.

### BRIEF DESCRIPTION OF THE FIGURE

FIG. 1 shows a schematic of the final construct of H22-Fel dI Ch1+Ch2 in pAcSAG-LIC.

### DETAILED DESCRIPTION OF THE INVENTION

It has now been found that the immunoreactivity of rFel dI for IgG and IgE antibody is improved dramatically by expressing the allergen in baculovirus.

Recombinant Fel dI, rFel dI Ch1+Ch2, in which the two chains are expressed in series and linked together by a glycine/serine linker (referred to herein as H22−), and CD64-targeted Fel dI (sFv22;Fel dI), which consists of the foregoing rFel d I Ch1+Ch2 linked to the sFv of monoclonal antibody H22 (mAb H22) (referred to herein as H22+) were genetically constructed. Mab H22 is the humanized anti-CD64 antibody (Graziano et al. J Immunol. 1995 155, 4996-5002). Since CD64 is only expressed by monocytes and dendritic cells, it is believed that the H22+ fusion protein targets Fel dI specifically to monocytes and dendritic cells via the sFv component, which is derived from the anti-CD64 monoclonal antibody H22. The molecular weight of the H22+ and H22− were 49 kd and 22 kd, respectively.

H22+ and H22− baculovirus expressed rFel dIs were purified by Ni affinity chromatography and compared with natural Fel dI (nFel dI) by ELISA using a panel of anti-Fel dI monoclonal antibodies and by RIA binding of the antigen to human IgE and IgG antibodies. Both H22+ and H22− rFel dI proteins demonstrated similar binding to nFel dI in ELISA using different combinations of monoclonal antibodies. Results from an ELISA are depicted in the following Table 1.

TABLE 1

| | nFel | H22 + FeldI | rFeldI | H22 + FeldI |
|---|---|---|---|---|
| Capture Ab | dI | Ch1 + Ch2 | Ch1 | Ch1 |
| 1G9 (EPI-B, CH1) | ++++ | ++++ | ++ | + |
| 8F3 (EBI-B, CH1) | + | + | − | − |
| 2H4 (EPI-C, CH2) | +++ | +++ | − | − |
| 10G7 (EPI-D, ?) | + | + | − | − |
| 11F5 (R&A, CH1) | − | − | − | − |
| 8H6 (R&A?, ?) | − | − | − | − |
| 6F9 (?, CH1) | ++++ | ++++ | ++++ | ++ |

The detection antibody in these studies was 3E4-biotin.

By inhibition RIA, H22+ rFel dI showed identical inhibition curves to nFel dI using IgG antibody in pooled sera from either Japanese (n=10) or US (n+6) cat allergic patients. The H22+ rFel dI inhibited binding of nfel dI by >95%. Excellent correlations were obtained by linear regression analysis comprising IgE antibody to H22+ rFel dI (n+155, r=0.72, p<0.001) or IgE antibody to H22− rFel dI (n=258, r=0.72, p<0.001) with nFel dI. These data show that IgG and IgE antibody binding by baculovirus expressed rFel dI is identical to nFel dI.

Accordingly, the baculovirus expressed rFel dIs of the present invention are believed to be useful in the diagnosis and treatment of cat allergy. Use of the rFel dI allergens of the present invention to diagnose a cat allergy in human serum samples is performed routinely in accordance with well known procedures. Similarly, incorporation of the allergens of the present invention into a treatment regime such as allergy shots for the treatment of cat allergies in humans is also performed in accordance with well known techniques.

The H22+ construct of the present invention is also useful in targeting of Fel dI to monocytes and dendritic cells for studies of antigen presentation and T cell responses in cat allergic patients.

The following nonlimiting examples are provided to further illustrate the present invention.

### EXAMPLES

### Example 1

Plasmids and Oligonucleotides

Baculovirus expression vector pAcSAG-LIC was purchased from Pharmingen. H22 sFv (encoding V_{H}V_{L} of the anti-CD64 antibody H22) was cloned from vector pJG225 (Medarex, Inc. Annandale, N.J., USA) into the BamHI and XbaI sites of pAcSAG-LIC and renamed pTJ225. Vectors pET11dΔHR chain-1 FeldI and pET11dΔHR chain-2 FeldI were provided by Immunologic (Waltham, Mass.). Chain 1 of FeldI was cloned into pTJ225 by PCR cloning. Chain 2 was cloned into vector pCR™2.1 of the TA cloning kit (Invitrogen, Carlsbad, Calif., USA). Primers were ordered from Integrated DNA Technologies (IDT, Coralville, Iowa.) and contained the following sequences:

```
Chain 1:
forward primer: 32 mer (SEQ ID NO:1)
5′ AGG ACT  CGA  GTG AAA TTT GCC CAG CCG TGA AG 3′
                    XhoI
backward primer: 36 mer (SEQ ID NO:2)
5′ TAA ACT TCG  CGG  CCG  C|CA  TAT  GAC ACA GAG GAC
TTG 3′ NotI NdeI
Chain 2:
forward primer: 28 mer (SEQ ID NO:3)
5′ GGG GCT  GCA  GGT CAA GAT GGC GGA AAC T 3′
                      PstI
backward primer: 33 mer (SEQ ID NO:4)
5′ GTT GTC AGC AGC  GGC  CGC TCT CCC CAA AGT GTT 3′
                                    NotI
```

Sequences complementary to the cDNA are shown in bold. To clone chain 1 and chain 2 succeedingly after H22, a linker oligo was designed. This linker oligo encodes the flexible peptide linker (Gly₄Ser)₃. Unique restriction sites were designed on both sides of the linker creating sticky ends immediately after annealing. The DNA sequence of the linker is described below.

Linker

sense, 54 mer (SEQ ID NO:5)
5′ TATG(GGT GGA GGA GGT TCT)ₓ₃CTGCA 3′
NdeI PstI
antisense, 48 mer (SEQ ID NO:6)
5′ G(AGAACCTCCTCCACC)ₓ₃CA 3′

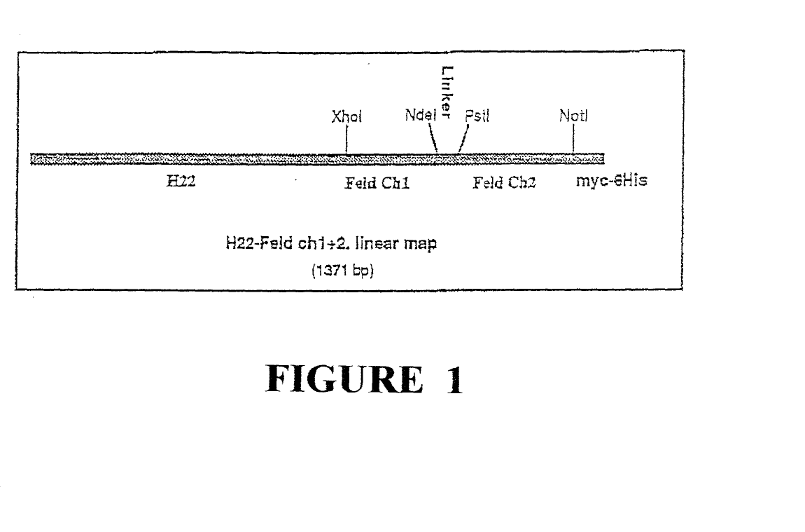

To generate H22-FeldI Ch1+Ch2 in baculovirus expression vector pAcSAG-LIC, FeldI Chl digested with XhoI and NdeI, linker with sticky ends NdeI and PstI and FeldI Ch2 restricted with PstI and NotI were ligated into the XhoI and NotI sites of pTJ225 in a four part ligation subcloning. The final construct is depicted in FIG. 1.

### Example 2

Generation of Recombinant Virus Containing the H22-FeldI Ch1+Ch2 Sequences

To generate recombinant virus, 3×10⁹ Sf9 cells in 60 mm tissue culture dish were co-transfected with 1 μg of baculovirus expression plasmid containing the genes of interest, using the transfection protocol according to the manufacturer's instructions. Four days after the transfection, the culture supernatant containing the recombinant viruses was collected. The titers of recombinant virus were then amplified to 5-10×10⁸ plaque forming units (pfu)/ml by infecting more Sf9 cells.

### Example 3

Protein Expression and Purification High FiveTM insect cells were chosen for large-scale production of recombinant protein. To determine the time course of recombinant protein expression, a monolayer of High FiveTM cells in a T-75 culture flask was infected with high titer recombinant virus at a multiplicity of infection (MOI) of 10. At specific intervals following infection, culture supernatant was collected and the proteins were precipitated with 72% trichloroacetic acid and 0.15% sodium deoxycholate. After resuspension in 0.1 volumes of sample buffer, SDS-PAGE (10-20% gradient gel) was performed and the gel was stained with Coomassie Blue R-250. Large scale expression was accomplished by infecting large volumes of suspension cultured cells. Cell-free supernatants were harvested 72 hours post-infection by removing the cells at 1000 rpm for 10 minutes at 4° C. At this time point expression of antibody fusion protein reached its peak in cell culture supernatants while there was limited intracellular protein resulting from cell lysis. The cell-free culture supernatants were then concentrated 10-fold, dialyzed and loaded onto a nickel (Ni)-affinity column (Novagen, Inc.). After washing the loading buffer, proteins were eluted with a linear gradient of imidazole in the same buffer. Fractions containing recombinant antibody-fusion protein were pooled and dialyzed. The pooled fractions were then applied to an anion-exchange column (Econo-Pac S-cartridge, Bio-Rad). the flow-through, containing recombinant protein, was collected and dialyzed in phosphate-buffered saline (PBS) The purity of all protein preparations was monitored by SDS-PAGE and was at least 95% homogenous. Protein concentrations were determined from A280 nm values calculated with molar extinction coefficient of 60293.0 A280 nm/mole. Yield was approximately 4-6 mg of purified recombinant protein per liter of Hi-5 culture supernatant.

What is claimed is:

1. A composition comprising a baculovirus expressed recombinant Fel dI.

2. The composition of claim 1 wherein the baculovirus expressed recombinant Fel dI comprises chain 1 and chain 2 expressed in series and linked together by a glycine/serine linker.

3. The composition of claim 2 further comprising a sFv of monoclonal antibody H22.

4. A method of diagnosing a human with cat allergy comprising contacting a serum sample from a human with a composition of claim 1 and determining the immunoreactive response of the serum sample to the composition of claim 1 wherein an immune reaction against the composition is indicative of an allergy to cats.

5. A method of protecting a human against a cat allergy comprising administering to a human a composition of claim 1.


SEQUENCE LISTING (6 sequences)

SEQ ID NO: 1 - DNA, 32 bp, Artificial Sequence
  Description of Artificial SequenceSynthetic
aggactcgagtgaaatttgcccagccgtgaag

SEQ ID NO: 2 - DNA, 36 bp, Artificial Sequence
  Description of Artificial SequenceSynthetic
taaacttcgcggccgccatatgacacagaggacttg

SEQ ID NO: 3 - DNA, 28 bp, Artificial Sequence
  Description of Artificial SequenceSynthetic
ggggctgcaggtcaagatggcggaaact

SEQ ID NO: 4 - DNA, 33 bp, Artificial Sequence
  Description of Artificial SequenceSynthetic
gttgtcagcagcggccgctctccccaaagtgtt

SEQ ID NO: 5 - DNA, 54 bp, Artificial Sequence
  Description of Artificial SequenceSynthetic
tatgggtggaggaggttctggtggaggaggttctggtggaggaggttctctgca

SEQ ID NO: 6 - DNA, 48 bp, Artificial Sequence
  Description of Artificial SequenceSynthetic
gagaacctcctccaccagaacctcctccaccagaacctcctccaccca